(19) United States
(12) Patent Application Publication
(10) Pub. No.: US 2007/0160554 A1
(43) Pub. Date: Jul. 12, 2007

(54) Acyl ribonucleosides and acyl deoxyribonucleosides, compositions of, and methods of making same

(75) Inventors: Peter Kempers, Grevenbroich (DE); Philippe Moussou, Nancy (FR); Louis Danoux, Saulxures Les Nancy (FR); Sabine Both, Duesseldorf (DE); Carsten Beverungen, Duesseldorf (DE)

Correspondence Address: COGNIS CORPORATION;PATENT DEPARTMENT, 300 BROOKSIDE AVENUE, AMBLER, PA, 19002, US

(73) Assignees: COGNIS IP MANAGEMENT GMBH, Dusseldorf (DE); COGNIS FRANCE S.A., Saint-Martory (FR)

(21) Appl. No.: 10/595,656

(22) PCT Filed: Oct. 23, 2004
(86) PCT No.: PCT/EP04/11990

§ 371 Date: Jan. 3, 2007

(30) Foreign Application Priority Data
Nov. 3, 2003 (EP) 03292735.2

(51) Int. Cl. A61K 31/7076; C07H 19/16; A61K 8/73

(52) U.S. Cl. 424/70.13; 514/47; 536/27.3

(57) ABSTRACT

The present invention is concerned with acyl ribonucleosides and with acyl deoxyribonucleosides. It is furthermore concerned with an enzymatic process for the manufacture of acyl ribonucleosides and of acyl deoxyribonucleosides. It is furthermore concerned with the use of acyl ribonucleosides and of acyl deoxyribonucleosides for cosmetic and for pharmaceutical purposes and with their use as a food supplement for humans or animals. It is furthermore concerned with compositions containing acyl ribonucleosides and acyl deoxyribonucleosides, whereby these compositions are suitable for cosmetic or for pharmaceutical purposes or as food supplement.

The present invention is concerned with acyl ribonucleosides and with acyl deoxyribonucleosides. It is furthermore concerned with an enzymatic process for the manufacture of acyl ribonucleosides and of acyl deoxyribonucleosides. It is furthermore concerned with the use of acyl ribonucleosides and of acyl deoxyribonucleosides for cosmetic and for pharmaceutical purposes and with their use as a food supplement for humans or animals. It is furthermore concerned with compositions containing acyl ribonucleosides and acyl deoxyribonucleosides, whereby these compositions are suitable for cosmetic purposes.

Ribonucleosides are known compounds in which a nucleobase is linked to the sugar D-ribose. In deoxyribonucleosides a nucleobase is linked to the sugar 2-desoxy-D-ribose. Nucleobases are in particular uracil, cytosine, thymine, adenine and guanine. The name of the ribonucleosides and deoxyribonucleosides is derived form the nucleobases. Depending on the sugar involved it is either uridine, cytidine, thymidine, adenosine and guanosine or deoxy-uridine, deoxy-cytidine, deoxy-thymidine, deoxy-adenosine and deoxy-guanosine.

The sugar moiety of the ribonucleosides has three OH-groups, the sugar moiety of the deoxyribonucleosides has two OH-groups. If at least one ofthese OH-groups is esterified with a carboxylic acid, acyl ribonucleosides and acyl deoxyribonucleosides are obtained. In the case of ribonucleosides one, two or three OH-groups can be esterified, in the case of deoxyribonucleosides only one or two OH-groups are present that can be esterified.

For the purpose of the present invention these O-acyl, di-O-acyl and tri-O-acyl compounds are all called acyl ribonucleoside or acyl deoxynucleoside. This includes the isomers that occur if only one or two of three OH-groups are esterified and if only one of two OH-groups groups is esterified. I. e. the term acyl ribonucleoside comprises the corresponding O-acyl, acyl, di-O-acyl and tri-O-acyl compounds (including isomers which are possible due to the different OH-groups that can be esterified) and mixtures of these compounds. E. g. for the purposes of the present invention stearoyl uridine comprises the three possible isomers of mono-o-stearoyl uridine, the three possible isomers of di-O-stearoyl uridine and tri-O-stearoyl uridine.

EP-B 0 339 075, WO 89/03837, US 20020035086 A1 and U.S. Pat. No. 6,258,795 B1 disclose acyl derivatives of uridine and cytidine and their use in pharmaceutical compositions. These documents and also U.S. Pat. No. 6,274,563 B1, U.S. Pat. No. 6,316,426 B1, U.S. Pat. No. 5,470,838 A1 and U.S. Pat. No. 5,583,117 A1 disclose the use of these acyl derivatives in pharmaceutical compositions for delivering exogenous uridine or cytidine to the tissue of an animal. The compositions disclosed are in the form of an oral suspension, a tablet, a dragee, an injectable solution or a suppository. These compositions are claimed to be useful for treating hepatopathies, diabetes, heart disease, cerebrovascular disorders, central nervous system disorders, Parkinson's disease, infant respiratory distress syndrome and for enhancement of phospholipid biosynthesis. The specific uridine or cytidine derivatives listed in the claims of these documents are their tri-O-acetyl derivatives, tri-O-propionyl derivatives and tri-O-butyryl derivatives.

None of these documents disclose the use of such acyl derivatives in cosmetics or the use of such acyl derivatives in compositions for topical applications to the human body or to the body of an animal.

EP-B 0 339 075, WO 89/03837,US 20020035086 A1 and U.S. Pat. No. 6,258,795 B1 disclose the acyl derivatives of uridine. In particular, mono-O-fatty acid derivatives and di-O-fatty acid derivatives are disclosed, wherein the fatty acids have 8 to 22 carbon atoms.

WO 2003057894 A1 discloses a process for manufacturing mono-O-acylated or di-O-acylated ribonucleosides by enzymatic selective hydrolysis of tri-O-acyl ribonucleoside.

The synthesis of 5′-O-acyl ribonucleosides by specific alcoholysis using lipases or proteases is also described by Nishino S. et al. in 1985 and by Zinni M. A. et al. in 2002 [S. Nishino, A. Rahman, H. Takamura, Y. Ishido, Tetrahedron 41, 1985, 5503-5506; M. A. Zinni, L. E. Iglesias, A. M. Iribarren, Regioselective preparation of 2′,3′-di-O-acyl ribonucleosides carrying lipophilic acyl groups through a lipase-catalysed alcoholysis, Biotechnology Letters 24, 2002, 979-983].

Selective direct enzymatic esterification of ribonucleosides was also described by other authors. Uemura et al. describe the enzymatic synthesis of O-acyl ribonucleosides using lipases in dimethyl acetamide (DMA), dimethyl formamide (DMF) or dimethyl sulfoxide (DMSO) as solvent, with acid anhydride as acyl donor. Under these conditions no reaction occurs with commercially available carboxylic acids and esters. Good yields (81 to 96%) were obtained with Pseudomonas fluorescens lipase [A. Uemura, K Nozaki, J.-I. Yamashita, M. Yasumoto. Tetrahedron Letters 30, 1989, 3817-3818].

Ozaki et al. describe the enzymatic esterification (acylation) of 5-fluorouridine or uridine with different lipases for the purpose of selective protection and subsequent chemical reaction. The reaction is carried out in dioxane. THF was also found to be a good solvent for the reaction. Selectivity of acylation is specific to each lipase. [S. Ozaki, K. Yamashita, T. Konishi, T. Maekawa, M. Eshima, A. Uemura, L. Ling, Enzyme aided regioselective acylation of nucleosides. Nucleosides & Nucleotides 14 (3-5), 1995,401-404; S. Ozaki, A. Uemura, T. Konishi, K. Yamashita, T. Maekawa, L. Ling, Enzyme aided regio-selective acylation and deacylation of nucleosides. Nucleic Acids Symposium Series 29, 1993, 53-54]. Singh H. K. et al. describe the enzymatic synthesis of 5′-O-acyl ribonucleosides using protease Subtilisin or crude protease Proleather in pyridine. The solvent has influence on the regioselectivity of the reaction. [H. K. Singh, G. L. Cote, T. M. Hadfield, Manipulation of enzyme regioselectivity by solvent engineering: enzymatic synthesis of 5′-O-Acyl ribonucleosides. Tetrahedron Letters 35 (9), 1994, 1353-1356]. The esterification of different sugars in DMF by protease Subtilisin was described with conversions from 57 to 85%, but long reaction times up to 7 days are required. The protease is not regioselective and synthesizes 5′-, 3′- and 2′-esters [S. Riva, J. Chopineau, A. P. G. Kieboom, A. M. Klibanov, Protease-Catalysed Regioselective Esterification of Sugars and Related Compounds in Anhydrous Dimethyl formamide. J. Am. Chem. Soc. 110, 1988, 584-589].

The problem underlying the present invention is the need for substances that can be used in cosmetic applications. There is a need for such substances improving appearance and aspect of human skin.

This problem is solved by the use of an acyl ribonucleoside or of an acyl deoxyribonucleoside having the following formulae I or II, 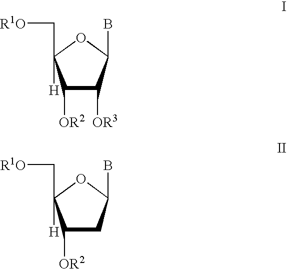
wherein
B is a nucleobase-moiety (preferably derived from uracil, cytosine, thymine, adenine or guanine),
R¹, R² and R³ are independently selected from the group consisting of
   a) hydrogen,
   b) a saturated or unsaturated, linear or branched acyl radical with 3 to 22 carbon atoms, optionally substituted with one or more substituents selected from the group consisting of hydroxy, hydroxy-alkyl, amino, amino-alkyl, mercapto, mercapto-alkyl, halogen and thiolanyl (for example palmitic acid, 16-hydroxyhexadecanoic acid, 12-hydroxystearic acid, 11-mercaptoundecanoic acid and thioctic acid (thioctic acid is alpha-lipoic acid or 1,2-dithiolane-3-pentanoic acid, CAS 6246-4 )),
   c) a saturated or unsaturated, linear or branched dicarboxylic acid radical with 3 to 22 carbon atoms (i. e. only one of the two carboxylic acid groups is esterified with one of the OH-groups of ribose or deoxyribose, the other —-COOH group is unmodified) or its derivative in which the —COOH-group that is not esterified with an OH-group of ribose or deoxyribose is replaced by —CONR′₂ or by CONR′₃⁺S³¹ (wherein R′ is a hydrogen atom, a saturated or unsaturated, linear or branched alkyl radical with 1 to 6 carbon atoms, or an aryl radical, or an aralkyl radical or an aralkylene radical and wherein S⁻ a counter ion (e. g.: Cl⁻ or an acetate ion)) or by —COHal (wherein Hal is a halogen atom) or by COSH (wherein S is sulphur) (for example hexadecanoic diacid or azelaic acid),
   d) a saturated or unsaturated, linear or branched dicarboxylic acid diradical with 3 to 22 carbon atoms (i. e. both carboxylic acid groups are esterified with an OH-group of ribose or deoxyribose) (for example hexadecanoic diacid or azelaic acid),
   e) an arylaliphatic acid radical and derivatives thereof, optionally substituted with one or more substituents selected from the group consisting of hydroxy, nitro, alkyl, alkoxy and halogen (for example cinnamic acid, phenylpropionic acid, caffeic acid (3,4-dihydroxycinnamic acid), ferulic acid (4-hydroxy-3-methoxycinnamic acid) and coumaric acid (4-hydroxycinnamic acid)) and
   f) a benzoic acid radical, optionally substituted with one or more substituents selected from the group consisting of hydroxy, nitro, alkyl, alkoxy and halogen (for example gallic acid (3,4,5-trihydroxybenzoic acid), vanillic acid (4hydroxy-3-methoxybenzoic acid) and protocatechuic acid (3,4-dihydroxybenzoic acid)) and wherein
      in the case of formula I at least one of the substituents R¹, R² and R³ is not hydrogen and in the case of formula II at least one of the substituents R¹ and R² is not hydrogen, for the manufacture of a cosmetic preparation or for the cosmetic treatment of the human body.

The acyl ribonucleosides and the acyl deoxyribonucleosides as defined in the previous paragraph are called by definition the acyl ribonucleosides and the acyl deoxyribonucleosides according to the present invention.

The two kinds of uses defined in the previous paragraph are a subject of the present invention. A further subject is the use as defined in the previous paragraph whereby the acyl ribonucleosides or the acyl deoxyribonucleosides that fall within the given definition are used in a composition that further comprises auxiliaries and/or additives which are common for cosmetic purposes.

The aforementioned compositions are a further subject of the present invention.

Only some of the acyl ribonucleosides and of the acyl deoxyribonucleosides according to the present invention are known in the state of the art. They are known as medicaments. Acyl ribonucleosides and acyl deoxyribonucleosides that are preferred for the cosmetic uses according to the present invention are the following compounds:

A compound selected from the group consisting of an acyl ribonucleoside and an acyl deoxynucleoside wherein the acyl group or the acyl groups is/are derived from a fatty acid, (preferably an unsubstituted, linear, saturated or unsaturated carboxylic acid) with 10 to 20 (preferably 16 to 18) carbon atoms or from 3-phenyl-propionic acid or from 12-hydroxy-stearic acid or from octadecanoic diacid or from hexadecanoic diacid or from azelaic acid or from octadecenoic diacid, whereby in the case of octadecanoic diacid orhexadecanoic diacid or azelaic acid or octadecenoic diacid one or both COOH groups of the acid can be esterified with a nucleoside.

Amongst these compounds the following compounds are especially preferred:

A compound selected from the group consisting of palmitoyl uridine, 5′-O-palmitoyl uridine, palmitoyl guanosine, palmitoyl adenosine, palmitoyl cytidine, oleyl uridine, 5′-O-oleyl uridine, oleyl guanosine, oleyl adenosine, oleyl cytidine, stearoyl uridine, 5′-O-stearoyl uridine, 3-phenyl-propionyl uridine, the monoester of uridine with octadecenoic diacid, the diester of uridine with octadecanoic diacid, the monoester of uridine with hexadecanoic diacid, the diester of uridine with hexadecanoic diacid, the monoester of uridine with octadecenoic diacid, the diester of uridine with octadecenoic diacid, the monoester of ulidine with azelaic acid, the diester of uridine with azelaic acid and 12-hydroxy-stearoyl uridine.

None of these preferred compounds is disclosed in the state of the art. They are especially useful for cosmetic applications because of their advantageous properties. What is shown in the documents of the state of the art is that acetyl, propanoyl and/or n-butanoyl derivatives of ribo or deoxyribonucleosides improve their bioavailability for a better delivering of exogenous ribo- or deoxyribonucleosides nucleosides to tissue. Compared to these derivatives, the selected preferred compounds have melanogenesis inhibition properties useful for cosmetic applications particularly to improve the appearance and aspect of human skin, for anti-ageing, whitening or lightening purposes.

Therefore these preferred compounds are a further subject of the present invention.

A further subject of the present invention is the use of the acyl ribonucleosides or of the acyl deoxyribonucleosides according to the present invention for the manufacture of a medicament for the treatment of human skin that has been damaged by UV-A radiation or by UV-B radiation or for the manufacture of a medicament for the treatment of inflammations of the human skin.

A further subject of the present invention is the use of the acyl ribonucleosides or of the acyl deoxyribonucleosides according to the present invention as a food supplement.

A further subject of the present invention is the use of the acyl ribonucleosides or of the acyl deoxyribonucleosides according to the present invention in orally administrable cosmetics.

The acyl ribonucleosides or of the acyl deoxyribonucleosides according to the present invention have the advantage that they protect (human) skin against ageing, against photo-ageing, that they can bring about a whitening effect to the skin and that they can remove pigmentation disorders.

The use of the acyl libonucleoside or the acyl deoxyribonucleoside according to the present invention as food supplement has the advantage that this leads to comparable effects as the application of these substances to the skin, i. e. protection of the skin against ageing or photo ageing, whitening the skin or remove pigmentation disorders. This type of application can be called “oral cosmetics”.

A further subject of the present invention is a process for manufacturing the acyl ribonucleosides or the acyl deoxyribonucleosides according to the present invention comprising reacting (optionally in a non-toxic solvent) the ribonucleoside or the deoxyribonucleoside with an acyl group donor in the presence of an enzymatic catalyst (optionally in soluble or in immobilised form).

This process is called the process according to the present invention.

In one embodiment of the process according to the present invention the acyl donor is the corresponding carboxylic acid.

In another embodiment of the process according to the present invention the acyl donor is also used as solvent.

In another embodiment of the process according to the present invention the solvent is selected from the group consisting of propan-2-ol, butan-2-ol, isobutanol, acetone, propanone, butanone, pentan-2-one, 1,2-ethanediol, 2,3-butanediol, 2-methylbutan-2-ol, tert-butanol, 2-methylpropanol, 4-hydroxy-2-methylpentanone, 4-hydroxy-4-methyl-2-pentanone, heptane, hexane and mixtures of two or more of these solvents.

In another embodiment of the process according to the present invention the molar ratio of the ribonucleoside or deoxyribonucleoside to the acyl donor is controlled during the reaction so that the ratio is always 0.01 to 20.00 (preferably between 0.02 and 10.00).

In another embodiment of the process according to the present invention additional amounts of ribonucleoside or deoxyribonucleoside, acyl donor, solvent, and/or enzymatic catalyst is added during the reaction.

In another embodiment of the process according to the present invention the resulting esters (the acyl ribonucleosides or the acyl deoxyribonucleosides according to the present invention) are purified by removing enzymatic particles and by removing the solvent.

In another embodiment of the present invention the process according to the present invention further comprises intermittently or continuously drawing off at least one constituent of the reaction medium.

In another embodiment of the process according to the present invention the temperature during the reaction is set to be from 20 to 100° C.

In another embodiment of the process according to the present invention the partial pressure above the reaction medium is set at from 10 mbar to 1000 mbar, and the reaction medium is subjected to agitation.

In another embodiment of the present invention the process according to the present invention further comprises eliminating residual ribonucleoside or deoxyribonucleoside or acyl donor by extraction with organic solvents, supercritical fluids, distillation, crystallization, adsorption or precipitation.

In another embodiment of the present invention the process according to the present invention further comprises fractionating of acyl ribonucleosides and/or deoxyribonucleosides produced by precipitation or chromatographic separation.

In another embodiment of the process according to the present invention the enzymatic catalyst is selected from the group consisting of a protease and a lipase. Preferably the protease or the lipase is immobilized on a carrier.

In another embodiment of the process according to the present invention water and/or alcohol is removed from the reaction medium by azeotropic distillation. Preferably the azeotrope is removed under total or nearly total reflux conditions through a distillation column.

In another embodiment of the process according to the present invention water and/or alcohol is removed from the reaction medium by molecular sieves which are contacted with the liquid reaction medium of with the gas phase that evaporates from the liquid reaction medium.

In another embodiment of the process according to the present invention water and/or alcohol is removed from the reaction medium by pervaporation in gas or liquid phases (pervaporation is a method of separation using membranes with vacuum as driving force).

One embodiment of the present invention is a compound selected from the group consisting of an acyl ribonucleoside and an acyl deoxynucleoside wherein the acyl group or the acyl groups is/are derived from a fatty acid, (preferably an unsubstituted, linear, saturated or unsaturated carboxylic acid) with 10 to 20 (preferably 16 to 18) carbon atoms or from 3-phenyl-propionic acid or from 12-hydroxy-stearic acid or from octadecanoic diacid or from hexadecanoic diacid or from azelaic acid or octadecenoic diacid, whereby in the case of octadecenoic diacid or azelaic acid or octadecenoic diacid one or both COOH groups of the acid can be esterified with a nucleoside. The manufacture and the use of this compound is an embodiment of the present invention, too.

One embodiment of the present invention is palmitoyl uridine, its manufacture and its use. Another embodiment of the present invention is 5′-O-palmitoyl uridine, its manufacture and its use. Another embodiment of the present invention is palmitoyl guanosine, its manufacture and its use. Another embodiment of the present invention is palmitoyl adenosine, its manufacture and its use. Another embodiment of the present invention is palmitoyl cytidine, its manufacture and its use. Another embodiment of the present invention is oleyl uridine, its manufacture and its use. Another embodiment of the present invention is 5′-O-oleyl uridine, its manufacture and its use. Another embodiment of the present invention is oleyl guanosine, its manufacture and its use. Another embodiment of the present invention is oleyl adenosine, its manufacture and its use. Another embodiment of the present invention is oleyl cytidine, its manufacture and its use. Another embodiment of the present invention is stearoyl uridine, its manufacture and its use. Another embodiment of the present invention is 5′-O-stearoyl uridine, its manufacture and its use. Another embodiment of the present invention is 3-phenyl-propionyl uridine, its manufacture and its use. Another embodiment of the present invention is 12-hydroxy-stearoyl uridine, its manufacture and its use. Another embodiment of the present invention is the monoester of uridine with octadecanoic diacid, its manufacture and its use. Another embodiment of the present invention is the diester of uridine with octadecanoic diacid, its manufacture and its use. Another embodiment of the present invention is the monoester of uridine with hexadecanoic diacid, its manufacture and its use. Another embodiment of the present invention is the diester of uridine with hexadecanoic diacid, its manufacture and its use. Another embodiment of the present invention is the monoester of uridine with azelaic acid, its manufacture and its use. Another embodiment of the present invention is the diester of uridine with azelaic acid, its manufacture and its use. Another embodiment of the present invention is the monoester of uridine with octadecenoic diacid, its manufacture and its use. Another embodiment of the present invention is the diester of uridine with octadecenoic diacid, its manufacture and its use.

The acyl derivatives of ribonucleosides or deoxyribonucleosides according to the present invention may be mono-O-acyl, di-O-acyl or tri-O-acyl derivatives of uridine, deoxy-uridine, pseudouridine, cytidine, deoxy-cytidine, thymidine, deoxy-thymidine, adenosine, deoxy-adenosine, guanosine, deoxy-guanosine. Pseudouridine (5-β-D-Ribofuranosyluracil, CAS 1445-07-4) is a natural ribonucleoside found in some plants.

A preferred acyl ribonucleoside is palmitoyl uridine. Another preferred acyl ribonucleoside is stearoyl uridine. Another preferred acyl ribonucleoside is 5′-O-palmitoyl-uridine. Another preferred acyl ribonucleoside is 5′-O-stearoyl-uridine. The combination of 5′-O-palrnitoyl-uridine and 5′-O-stearoyl-uridine is also a preferred embodiment of the present invention.

A preferred cosmetic use of the acyl ribonucleosides or of the acyl deoxyribonucleosides according to the present invention is their cosmetic use to prevent and/or to fight against skin ageing, ageing caused by exogenous factors and/or photo-ageing.

A preferred cosmetic use of the acyl ribonucleosides or of the acyl deoxyribonucleosides according to the present invention is their cosmetic use for the inhibition of melanin synthesis in hair and/or skin cells, as whiteners or lighteners, and/or to fight against pigmentation disorders.

One embodiment of the present invention is the use of the acyl ribonucleosides and/or the acyl deoxyribonucleosides according to the present invention in cosmetic, dermopharmaceutical or food formulations, especially to fight against skin ageing and photo ageing, to fight against pigmentation disorders and to whiten the skin.

One embodiment of the present invention is an enzymatic process to synthesize O-acyl ribonucleosides and/or O-acyl deoxyribonucleosides (in this context o-acyl means O-acyl, di-O-acyl and tri-O-acyl) with good yields under mild conditions and using solvents that are compatible with the use of the reaction products in cosmetic applications or food applications.

The processes according to the state of the art use DMF, THF, pyridine, dioxane, DMA or DMSO as solvent. This is a disadvantage, because these solvents are unfavourable if the reaction products are used for cosmetic applications, as medicaments or as food supplements (traces of the solvent can be present in the product as impurity).

The process according to the present invention has the advantage that it does not use any hazardous solvent. Thus the process according to the present invention reduces the complex post-synthesis purifying operations to remove solvents.

In the human epidermis and dermis, chronological ageing causes for examples structural damages as dryness, roughness, formation of dryness wrinkles. Exogenous factors such as UV light, chemicals, oxidants or environmental pollutants can have a cumulative effect and accelerate or enhance endogenous this ageing processes. In the epidermis and dermis, these exogenous factors cause some visible vascular dilatations as couperosis, formation of wrinkles, local hyperpigmentation, abnormal pigmentation as age spots, increased susceptibility to mechanical stress.

The acyl ribonucleosides and the acyl deoxyribonucleosides according to the present invention have advantages when used in cosmetic or dermopharmaceutical compositions (or compositions for oral administration). They care of the naturally aged skin, as well as fight against and/or prevent the damages of intrinsic ageing, ageing caused by exogenous factors and/or photo ageing as described above.

Moreover, the acyl ribonucleosides and the acyl deoxyribonucleosides according to the present invention inhibit the synthesis of melanin. Consequently, they can be used for the inhibition of melanin synthesis in hair and skin cells, to whiten or lighten the sldn. They may be also used to prevent or to fight against local hyperpigmentation or abnormal pigmentation as age spots.

The amount of the acyl ribonucleosides and the acyl deoxyribonucleosides according to the present invention in the compositions according to the present invention preferably ranges from 0.0001 to 10%, more, more preferably from 0.01 to 5% by weight.

The acyl ribonucleosides and the acyl deoxyribonucleosides according to the present invention may be synthesized using well-known chemical acylation processes from the state of the art. Acyl donors may be chosen from the group consisting of carboxylic acids of the formula RCOOH, the halogen derivatives of these acids RCOHal, anhydrides ofthe formula RCOOCR or esters of the formula RCOOR′ wherein R′ is a C₁-C₆ alkyl group, and wherein R is chosen in such a way that the resulting product is an acyl ribonucleoside or an acyl deoxyribonucleoside according to the present invention.

The reaction may be carried out in a (preferably anhydrous) solvent under inert atmosphere. The solvent may be selected from the group consisting of toluene, pyridine, chloroform, tetrahydrofurane and acetone.

The process according to the present invention is an enzymatic synthesis which can be carried out under milder conditions than the chemical syntheses or enzymatic processes known in the state of the art, thus avoiding the use of toxic solvents like pyridine, benzene, DMF, THF, dioxane, and/or high temperatures, and/or the production of by-products as salts or products of the degradation of ribonucleosides and/or deoxyribonucleosides, which have to be removed by additional purification steps.

One embodiment of the present invention is a method for the enzymatic synthesis of O-acyl ribonucleosides and/or O-acyl deoxyribonucleosides, wherein the reaction is carried out in a non-toxic solvent that can totally or partially solve the selected ribonucleoside or deoxyribonucleoside and acyl donors. The solvent or solvents may in particular be selected from the group consisting of the acyl-donor used, propan-2-ol, butan-2-ol, isobutanol, acetone, propanone, butanone, pentan-2-one, 1,2-ethanediol, 2,3-butanediol, 2-methylbutan-2-ol, tert-butanol, 2-methylpropanol and 4-hydroxy-2-methylpentanone, 4-hydroxy-4-methyl-2-pentanone, aliphatic hydrocarbons such as heptane, hexane and a mixture of two or more of these solvents.

The process according to the present invention can be carried out in the following way. Introducing predetermined amounts of a ribonucleoside and/or a deoxyribonucleoside in a reactor together with the solvent so that a reaction medium is formed, adding an acyl donor and an enzymatic catalyst, carrying out the reaction under conditions allowing to eliminate the water and/or alcohol formed during the reaction. This water and/or alcohol may be removed under vacuum, by adsorption on molecular sieves, by distillation, including azeotropic distillation, or with membranes e.g. by pervaporation. This reaction can be conducted in batch mode, or continuous mode, or also in fed-batch with one or more substrates. Preferably, the removal of water/alcohol is carried out by azeotropic distillation with a solvent that forms an azeotrope with water/alcohol. More preferably, the azeotrope is collected in a column under total or nearly total reflux conditions. Finally, the resulting O-acyl ribonucleosides or O-acyl deoxyribonucleosides are purified at least by separating the enzymatic particles (for example by decanting, filtering or centrifuging) and the solvent (for example by evaporation, distilling or membrane filtration).

The reaction can be conducted in such a way that the inhibition or the deactivation of the enzymatic reaction which is observed in the presence of strong concentrations of ribonucleoside and/or deoxyribonucleoside, acyl donors, alcohol and/or water accumulation is initially limited. Substrates may be introduced gradually in a controlled manner in the course of the reaction to avoid reaching concentration levels which would inhibit the enzyme reaction.

The reaction may be conducted in such a way that the ribonucleosides or deoxyribonucleosides/acyl donors molar ratio is from 0.01 to 20.00, preferably from 0.02 to 10.00. To optimise the running of the synthesis reaction, it is possible to proceed by intermittently or continuously drawing off at least one constituent of the reaction medium. The constituent(s) drawn off could possibly be returned to the reactor after being fractionated.

The reaction vessel or reactor used for carrying out the method of the invention is advantageously equipped with a temperature control, water and/or alcohol control and a pressure control, means for adding reagents and means for drawing off products.

While the synthesis reaction is in process the temperature is advantageously set at from 20 to 100° C., the partial pressure above the reaction medium is advantageously set from 10 mbar (1000 Pa) to 1000 mbar (100000 Pa), and the reaction medium is advantageously subjected to gentle agitation.

To obtain preparations resulting in acyl ribonucleoside or acyl deoxyribonucleoside of high purity, provision may further be made to carry out additional final fractionating operations, for example through removal of the residual ribonucleoside or deoxyribonucleoside or acyl donor by extraction with organic solvents, supercritical fluids, distillation or molecular distillation, chromatographic separation, precipitation or crystallisation.

The ribonucleoside and deoxyribonucleosides used in the invention can comprise any compound chosen from the group consisting of uridine, deoxy-uridine, pseudouridine, cytidine, deoxy-cytidine, thymidine, deoxy-thymidine, adenosine, deoxy-adenosine, guanosine and deoxy-guanosine.

The acyl donor may be chosen from known fatty acids or their methyl, ethyl, propyl or butyl esters, or their triglycerides. This fatty acid may preferably be selected from the group consisting of a straight or branched aliphatic acid, saturated, unsaturated or cyclic containing from 3 to 22 carbon atoms, optionally substituted with one or more substituents selected from the group consisting of hydroxy, amino, mercapto, halogen, thiolanyl (for example palmitic acid, 16-hydroxyhexadecanoic acid, 12-hydroxystearic acid, 11-mercaptoundecanoic acid, thioctic acid), a straight or branched aliphatic diacid, saturated, unsaturated containing from 3 to 22 carbon atoms (for example hexadecanedioic acid, azelaic acid), an arylaliphatic acid and a derivative thereof, optionally substituted with one or more substituents selected from the group consisting of hydroxy, nitro, alkyl, alkoxy and halogen atoms (for example phenyl-propionic acid, cinnamic acid, caffeic acid (3,4-dihydroxycinnamic acid), ferulic acid (4-hydroxy-3-methoxycinnamic acid), coumaric acid (4-hydroxycinnamic acid)), a benzoic acid optionally substituted with one or more substituents selected from the group consisting of hydroxy, nitro, alkyl, alkoxy and (halogen atoms for example gallic acid (3,4,5-trihydroxybenzoic acid), vanillic acid (4-hydroxy-3-methoxybenzoic acid), protocatechuic acid (3,4-dihydroxybenzoic acid)). The fatty acid esters may preferably be selected from the methyl or ethyl esters of the above compounds.

The enzymatic catalyst used must of course cause and encourage transfer of an acyl group from an acyl donor to a ribonucleoside or deoxyribonucleoside, and may advantageously comprise a protease or lipase, e.g. from Thermomyces, Candida cylindracea, Candida lipolytica, Candida rugosa, Candida antarctica A, Candida antarctica B, Candida utilis, Chromobacterium viscosum, Geotrichum viscosum, Geotrichum candidum, Mucor javanicus, Mucor miehei, porcine pancreas, Pseudomonas species, Pseudomonas fluorescens, Pseudomonas sepacia, Rhizomucor meihei, Rhizopus arrhizus, Rhizopus delemar, Rhizopus delemar, Rhizopus niveus, Rhizopus oryzae, Aspergillus niger, Penicillium roquefortii, Penicillium cambertii, Pseudomonas fluorescens or from an esterase of Bacillus sp., Bacillus thermoglucosidasius, Mucor miehei, horse liver, Saccharomyces cerevisiae, pig liver.

The enzymes may be used individually or in combination of more than one enzyme. An enzymatic catalyst may be used in its free form, or immobilized on an inert support, so that it can be recycled. Lipases derived from Mucor miehei and Aspergillus niger are preferably used. In particular, Lipozyme® TL IM (Thermomyces lanuginosus lipase immobilized), Lipozyme® RM IM (Rhizomucor miehei lipase immobilized), Novozym® 735 L (Candida antarctica B lipase, free), Novozym® 525 L (Candida antarctica B lipase, free) and/or Novozym® 435 (Candida antarctica B lipase immobilized), which are all products of Novozymes A/S, Denmark, can be used. The enzymes are preferably used in quantities of 0.01 to 15% by weight, preferably 1 to 10% by weight, based on the amount of ribonucleosides or deoxyribonucleosides.

Cosmetic treatment of the human body according to the present invention comprises the treatment of skin and/or hairs and/or skin appendices. Skin appendices means nails, sebaceous glands, sweat glands etc.

The auxiliaries and additives which are common for cosmetic purposes can be selected from the group consisting of oily bodies, surfactants, emulsifiers, fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, film formers, antidandruff agents, swelling agents, insect repellents, hydrotropes, solubilizers, preservatives, perfume oils and dyes.

In one embodiment of the present invention the auxiliaries and additives which are common for cosmetic purposes are selected from the group consisting of surfactants, emulsifiers, fats, waxes, stabilizers, deodorants, antiperspirants, antidandruff agents and perfume oils.

The total content of auxiliaries and additives may be 1 to 50% by weight, preferably 5 to 40% by weight, based on the cosmetic and/or pharmaceutical preparations. The preparations can be prepared by customary cold or hot processes; preference is given to using the phase-inversion temperature method.

For the purposes of the invention, cosmetic preparations can mean care agents. Care agents are understood as meaning care agents for skin and hair. These care agents include, inter alia, cleansing and restorative action for skin and hair.

Application can be topical or oral in the form of tablets, dragees, capsules, juices, solutions and granules.

The compositions and cosmetic preparations according to the invention can be used for the preparation of cosmetic and/or dermopharmaceutical preparations, e. g. hair shampoos, hair lotions, foam baths, shower baths, creams, gels, lotions, alcoholic and aqueous/alcoholic solutions, emulsions, wax/fat compositions, stick preparations, powders or ointments. Furthermore, the preparations for oral application according to the invention can also be incorporated into tablets, dragees, capsules, juices, solutions and granules.

These preparations can also comprise, as further auxiliaries and additives which are common for cosmetic purposes, oily bodies, surfactants, emulsifiers, fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, antidandruff agents, film formers, swelling agents, insect repellents, hydrotropes, solubilizers, preservatives, perfume oils, dyes and other auxiliaries and additives which are common for cosmetic purposes.

Surfactants (or Surface-active substances) that may be present are anionic, nonionic, cationic and/or amphoteric or amphoteric surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefm sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylaniino acids, e. g. acyl lactylates, acyl tartrates, acyl glutamates and acyl aspartates, alkyl oligoglucoside sulfates, protein fatty acid condensates (in particular wheat-based vegetable products) and alkyl (ether) phosphates. If the anionic surfactants contain polyglycol ether chains, these may have a conventional homologous distribution, but preferably have a narrowed homologous distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. If the nonionic surfactants contain polyglycol ether chains, these may have a conventional homologous distribution, but preferably have a narrowed homologous distribution. Typical examples of cationic surfactants are quaternary ammonium compounds, e.g. dimethyldistearylammonium chloride, and ester quats, in particular quaternized fatty acid trialkanolamine ester salts. Typical examples of amphoteric or zwitterionic surfactants are alkylbetaines, alkylamidobetaines, aminopropionates, aminoglycinates, imidazolinium-betaines and sulfobetaines. Said surfactants are known compounds. With regard to structure and preparation of these substances, reference may be made to relevant review works.

Typical examples ofparticularly suitable mild, i.e. particularly skin-compatible surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter preferably based on wheat proteins.

Suitable oily bodies are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters ofbranched C₆-C₁₃-carboxylic acids with linear or branched C₆-C₂₂-fatty alcohols, for example myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxycarboxylic acids with linear or branched C₆-C₂₂-fatty alcohols, in particular dioctyl malates, esters of linear and/or branched fatty acids with polyhydric alcohols (for example propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, for example dicaprylyl carbonates (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, for example dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, for example squalane, squalene or diaLkylcyclohexanes.

Suitable emulsifiers are, for example, nonionogenic surfactants from at least one of the following groups:
addition products of from 2 to 30 mol of ethylene oxide and/or 0to 5 mol ofpropylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical;
alkyl and/or alkenyl oligoglycosides having 8 to 22 carbon atoms in the alk(en)yl radical and the ethoxylated analogs thereof;
addition products of from 1 to 15 mol of ethylene oxide onto castor oil and/or hydrogenated castor oil;
addition products of from 15 to 60 mol of ethylene oxide onto castor oil and/or hydrogenated castor oil;
partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide;
partial esters of polyglycerol (average degree of self-condensation 2 to 8), polyethylene glycol (molecular weight 400 to 5 000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;
mixed esters of pentaeryhritol, fatty acids, citric acid and fatty alcohols and/or mixed esters of fatty acids having 6 to 22 carbon atoms, methylglucose and polyols, preferably glycerol or polyglycerol,
mono-, di- and trialkyl phosphates, and mono-, di- and/or tri-PEG alkyl phosphates and salts thereof;
wool wax alcohols;
polysiloxane-polyalkyl-polyether copolymers and corresponding derivatives;
block copolymers, e.g. polyethylene glycol-30 dipolyhydroxystearates;
polymer emulsifiers, e.g. Pemulen® grades (TR-1, TR-2) from Goodrich;
polyalkylene glycols, and
glycerol carbonate.

The addition products of ethylene oxide and/or of propylene oxide onto fatty alcohols, fatty acids, alkylphenols or onto castor oil are known, commercially available products. These are homologous mixtures whose average degree of alkoxylation corresponds to the ratio of the amounts of ethylene oxide and/or propylene oxide and substrate with which the addition reaction is carried out. C_{12/18}-fatty acid mono- and diesters of addition products of ethylene oxide onto glycerol are known as refatting agents for cosmetic preparations.

Alkyl and/or alkenyl oligoglycosides, their preparation and their use are known from the prior art. They are prepared, in particular, by reacting glucose or oligosaccharides with primary alcohols having 8 to 18 carbon atoms. With regard to the glycoside radical, both monoglycosides, in which a cyclic sugar radical is glycosidically bonded to the fatty alcohol, and also oligomelic glycosides having a degree of oligomerization of up to, preferably, about 8, are suitable. The degree of oligomerization here is a statistical average value that is based on a homologous distribution customary for such technical-grade products.

Typical examples of suitable partial glycerides are hydroxy stearic acid monoglyceride, hydroxy stearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linoleic acid monoglyceride, linoleic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride, and the technical-grade mixtures thereof which may also comprise small amounts of triglyceride as a minor product of the preparation process. Likewise suitable are addition products of 1 to 30 mol, preferably 5 to 10 mol, of ethylene oxide onto said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise suitable are addition products of 1 to 30 mol, preferably 5 to 10 mol, of ethylene oxide onto said sorbitan esters.

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants can be used as emulsifiers. The term “zwitterionic surfactants” refers to those surface-active compounds that carry at least one quaternary ammonium group and at least one carboxylate and one sulfonate group in the molecule. Particularly suitable zwitterionic surfactants are the betaines, such as N-alkyl-N,N-dimethylammonium glycinates, for example cocoalkyldimethylammonium glycinate, N-acylaminopropyl-N,N-dimethylammonium glycinates, for example cocoacylamino-propyldimethylammonium glycinate, and 2-alkyl-3-carboxymethyl-3-hydroxy-ethylimidazolines having in each case 8 to 18 carbon atoms in the alkyl or acyl group, and cocoacylaminoethylhydroxyethylcarboxymethyl glycinate. Particular preference is given to the fatty acid amide derivative known under the CTFA name Cocamidopropyl Betaine. Likewise suitable emulsifiers are ampholytic surfactants. The term “ampholytic surfactants” means those surface-active compounds that, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyl-taurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkyl aminopropionate, cocoacylaminoethyl amino-propionate and C_{12/18}-acylsarcosine. Finally, cationic surfactants are also suitable emulsifiers, those of the ester quat type, preferably methyl-quaternized difatty acid triethanolamine ester salts, being particularly preferred.

Fats and waxes that can be used are described in the following text. Typical examples of fats are glycerides, i.e. solid or liquid vegetable or animal products which consist essentially of mixed glycerol esters of higher fatty acids, suitable waxes are inter alia natural waxes, for example candelilla wax, carnauba wax, japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), for example montan ester waxes, sasol waxes, hydrogenated jojoba waxes, and synthetic waxes, for example polyalkylene waxes and polyethylene glycol waxes. In addition to the fats, suitable additives are also fat-like substances, such as lecithins and phospholipids. The term lecithins is understood by the person skilled in the art as meaning those glycerophospholipids which form from fatty acids, glycerol, phosphoric acid and choline by esterification. Lecithins are thus frequently also [lacuna] as phosphatidylcholines (PC). Examples of natural lecithins which maybe mentioned are the cephalins, which are also referred to as phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerophosphates), which are generally considered to be fats. In addition, sphingosines and sphingolipids are also suitable.

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, for example fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying agents and thickeners that can be used are described in the following text. Suitable bodying agents are primarily fatty alcohols orhydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and Tyloses, carboxymethylcellulose and hydroxyethylcellulose, and also relatively high molecular weight polyethylene glycol mono- and diesters of fatty acids, polyacrylates (e.g. Carbopols® and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone, surfactants, for example ethoxylated fatty acid glycerides, esters of fatty acids with polyols for example pentaerythritol or trimethylolpropane, fatty alcohol ethoxylates having a narrowed homolog distribution or alkyl oligoglucosides, and electrolytes such as sodium chloride and ammonium chloride.

Superfatting agents which can be used are substances for example lanolin and lecithin, and polyethoxylated or acylated lanolin and lecithin derivatives, polyol fatty acid esters, monoglycerides and fatty acid alkanolamides, the latter also serving as foam stabilizers.

Stabilizers which can be used are metal salts of fatty acids, for example magnesium, aluminum and/or zinc stearate or ricinoleate.

Polymers that can be used are described in the following text. Suitable cationic polymers are, for example, cationic cellulose derivatives, for example a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, for example Luviquat® (BASF), condensation products of polyglycols and amines, quaternized collagen polypeptides, for example lauryldimonium hydroxypropyl hydrolyzed collagen (Lamequat®L/Grünau), quaternized wheat polypeptides, polyethyleneimine, cationic silicone polymers, for example amodimethicones, copolymers of adipic acid and dimethylaminohydroxypropyl-diethylenetriamine (Cartaretins®/Sandoz), copolymers of acrylic acid with dimethyl-diallylammonium chloride (Merquat® 550/Chemviron), polyaminopolyamides and crosslinked water-soluble polymers thereof, cationic chitin derivatives, for example quatemized chitosan, optionally in microcrystalline dispersion, condensation products from dihaloalkyls, for example dibromobutane with bisdialkylamines, for example bis-dimethylamino-1,3-propane, cationic guar gum, for example Jaguar® CBS, Jaguar® C-17, Jaguar® C-16 from Celanese, quaternized ammonium salt polymers, for example Mirapol® A-15, Mirapol® AD-1, Mirapol® AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamidopropyltrimethylammonium chloride-acrylate copolymers, octylacrylamide-methyl methacrylate-tert-butylaminoethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones.

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are be liquid or in resin form at room temperature. Also suitable are simethicones, which are mixtures of dimethicones having an average chain length of from 200 to 300 dimethyl-siloxane units and hydrogenated silicates.

Deodorants and antimicrobial agents that can be used are described in the following text. Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents. Suitable antimicrobial agents are, in principle, all substances effective against gram-positive bacteria, for example 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chloro-phenoxy)-1,2-propanediol, 3-iodo-2-propynyl butylcarbamate, chlorohexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, for example n-octylsalicylamide or n-decylsalicylamide.

Suitable enzyme inhibitors are preferably, for example, esterase inhibitors. These are preferably trialkyl citrates, such as trimethyl citrate, tripropyl citrate, triisopropyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, for example lanosterol, cholesterol, campesterol, stigmasterol and sitosterol sulfate or phosphate, dicarboxylic acids and esters thereof, for example glutaric acid, monoethyl glutarate, diethyl glutarate, adipic acid, monoethyl adipate, diethyl adipate, malonic acid and diethyl malonate, hydroxycarboxylic acids and esters thereof, for example citric acid, malic acid, tartaric acid or diethyl tartrate, and zinc glycinate.

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, for example extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, for example civet and castoreum. Typical synthetic fragrance compounds are products of the ester, ether, aldehyde, ketone, alcohol and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, a-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evemyl, iraldein gamma, phenylacetic acid, geranyl acetate, benzyl acetate, rose oxide, romilat, irotyl and floramat alone or in mixtures.

Antiperspirants reduce the formation of perspiration by influencing the activity of the eccrine sweat glands, thus counteracting underarm wetness and body odor. Aqueous or anhydrous formulations of antiperspirants typically comprise one or more of the following ingredients: astringent active ingredients, oil components, nonionic emulsifiers, coemulsifiers, bodying agents, auxiliaries, for example thickeners or complexing agents, and/or nonaqueous solvents, for example ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium penta-chlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be anti-inflammatory, skin-protective or perfumed ethereal oils, synthetic skin-protective active ingredients and/or oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, for example xanthan gum, hydroxyethylcellulose, polyvinylpyrrolidone or high molecular weight polyethylene oxides.

Film formers that can be used are described in the following text. Customary film formers are, for example, chitosan, microcrystalline chitosan, quaternized chitosan, polyvinyl-pyrrolidone, vinylpyrrolidone-vinyl acetate copolymers, polymers of the acrylic acid series, quaternary cellulose derivatives, collagen, hyaluronic acid and salts thereof, and similar compounds.

Suitable antidandruff active ingredients are piroctone olamine (1-hydroxy-4-methyl-6-(2,4,4-trimethylpentyl)-2-(1H)-pyridinone monoethanolamine salt), Baypival® (climbazole), Ketoconazole®, (4-acetyl-1-{-4-[2-(2,4-dichlorophenyl) r-2-(1H-imidazol-1-ylmethyl)-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur tar distillates, salicyclic acid (or in combination with hexachlorophene), undecylenic acid monoethanolamide sulfosuccinate Na salt, Lamepon® UD (protein undecylenic acid condensate), zinc pyrithione, aluminum pyrithione and magnesium pyrithione/dipyrithione magnesium sulfate.

The swelling agents for aqueous phases may be montmorillonites, clay mineral substances, Pemulen, and alkyl-modified Carbopol grades (Goodrich).

Suitable insect repellents are N,N-diethyl-m-toluamide, 1,2-pentanediol or ethyl butylacetylaminopropionate.

To improve the flow behavior, hydrotropes, for example ethanol, isopropyl alcohol, or polyols, can be used. Polyols which are suitable here preferably have 2 to 15 carbon atoms and at least two hydroxyl groups. The polyols can also contain further functional groups, in particular amino groups, or be modified with nitrogen. Typical examples are:
glycerol;
alkylene glycols, for example, ethylene glycol, diethylene glycol, propylene glycol, butylene glycol, hexylene glycol, and polyethylene glycols with an average molecular weight of from 100 to 1 000 daltons;
technical-grade oligoglycerol mixtures with a degree of self-condensation of from 1.5 to 10, for example, technical-grade diglycerol mixtures with a diglycerol content of from 40 to 50% by weight;
methylol compounds, such as trimethylolethane, trimethylolpropane, trimethylol-butane, pentaerythritol and dipentaerythritol;
lower alkyl glucosides, in particular those with 1 to 8 carbon atoms in the alkyl radical, for example methyl and butyl glucoside;
sugar alcohols with 5 to 12 carbon atoms, for example sorbitol or mannitol,
sugars with 5 to 12 carbon atoms, for example glucose or sucrose;
amino sugars, for example glucamine;
dialcohol amines, such as diethanolamine or 2-amino-1,3-propanediol.

Suitable preservatives are, for example, phenoxyethanol, formaldehyde solution, parabenes, pentanediol or sorbic acid, and the other classes of substance listed in Annex 6, Part A and B of the Cosmetics Directive.

Perfume oils which may be used are preferably mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pine wood, sandalwood, guaiac wood, cedarwood, rosewood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, for example civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydro-myrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evemyl, iraldein gamma, phenylacetic acid, geranyl acetate, benzyl acetate, rose oxide, romilat, irotyl and floramat alone or in mixtures.

Dyes which can be used are the substances which are approved and suitable for cosmetic purposes. These dyes are normally used in concentrations of from 0.001 to 0.1% by weight, based on the total mixture.

### EXAMPLES

The enzymes used in the examples are commercially available enzymes. Novozyme® and Lipozyme® are obtainable from Novozymes A/S, Denmark. Novozym® 735 L is Candida antarctica B lipase, free (i. e. not immobilized). Novozym® 435 is Candida antarctica B lipase immobilized. Lipozyme® RM IM is Rhizomucor miehei lipase immobilized. Lipozyme® TL IM is Thermomyces lanuginosus lipase immobilized. Lipase AY is obtainable from the company Amano. Lipomod 34 is obtainable from the company Biocatalysts.

The degree of acylation of the compounds obtained in the examples is one.

### Example 1

### Synthesis of Palmitoyl Rribonucleoside with Different Lipases

The synthesis of acyl ribonucleoside was performed with different lipases. 2.5 g uridine (10 mmol) was esterified with 5.25 g palmitic acid (20 mmol) in 30 ml of 2-methyl-2-butanol with 0.5 g of different immobilized lipases. Reactions were performed in shaked flasks at 60° C. with addition of 3.5 g of molecular sieves for 67 hours. Conversion was calculated on the amount of fatty acid consumed.

| Lipase | Source of enzyme | Conversion [%] |
|---|---|---|
| Novozym 735 | Candida antarctica A | 41.5 |
| Novozym 435 | Candida antarctica B | 95.3 |
| Lipozyme RM | Rhizomucor miehei | |
| IM | | 13.3 |
| Lipozyme TL IM | Thermomyces lanuginosus | 22.1 |
| Lipase AY | Candida rugosa | 7.3 |
| Lipomod 34 | Candida cylindracea | 8.4 |

### Example 2

### Synthesis of Palmitoyl Ribonucleosides from Different Nucleosides

The synthesis of different acyl ribonucleosides was carried out as follows: 1 g nucleoside (4 mmol uridine, 3.5 mmol guanosine, 4 mmol cytidine and 3.7 mmol adenosine) was esterified with twice the molar amount of palmitic acid (7-8 mmol). Conversions were carried out with 0.2 g Novozym 435 as catalyst in shaked flasks with 20 ml 2-methyl-2-butanol at 60° C. with addition of 2 g of molecular sieves for 68 hrs. Conversion was calculated on the amount of fatty acid consumed.

| Nucleosides | Conversion [%] |
|---|---|
| Uridine | 59.2 |
| Guanosine | 25.4 |
| Cytidine | 55.8 |
| Adenosine | 68.4 |

### Example 3

### Synthesis of Stearoyl Ribonucleoside in Different Solvents

The synthesis of acyl ribonucleoside was performed in different solvents. 1 g uridine (4 mmol) was esterified with 2.3 g stearic acid (8 mmol) in 20 ml of different solvents with 0.2 g Novozym 435 as biocatalyst. Reactions were performed in shaked flasks at 60° C. with addition of 2 g of molecular sieves for 68 hrs. Conversion was calculated on the amount of fatty acid consumed.

| Solvent | Conversion [%] |
|---|---|
| 2-Methyl-2-butanol | 51.0 |
| Acetone | 88.5 |
| Hexane | 55.3 |
| t-Butanol | 97.0 |
| Ethyl-methyl-ketone | 92.1 |

### Example 4

### Synthesis of Acyl Uridine with Different Acyl Donors

The synthesis of uridine esters (acyl uridines) was performed with different acyl donors. 1 g uridine (4 mmol) was esterified with 8 mmol of 3-phenylpropionic acid, octadecanoic diacid, octadecanoic diacid or azelaic acid in 20 ml 2-methyl-2-butanol with 0.2 g Novozym 435 as biocatalyst. Reactions were performed in shaked flasks at 60° C. for 68 hrs with addition of 2 g of molecular sieves. Conversion was calculated on the amount of acyl donor consumed.

| Acids | Conversion [%] |
|---|---|
| 3-Phenylpropionic acid | 56.4 |
| Octadecanoic diacid | 51.4 |
| Octadecanoic diacid | 57.8 |
| Azelaic acid | 63.7 |

### Example 5

### Synthesis of 12-hydroxystearoyl Uridines with Different Ratios of Acyl Donor to Uridine.

The synthesis of uridine esters was performed with different amounts of acyl donors. 0,5 g uridine (2 mmol) was esterified with different amounts of 12-hydroxystearic acid (2-10 mmol) (HAS) in 20 ml of t-butanol with 1 g Novozym 435 as biocatalyst. Reactions were performed in shaked flasks for 48 hrs at 60° with addition of 3 g of molecular sieves. Conversion was calculated based on HPLC analysis.

| Molar ratio Uridine/12-HSA | Conversion [%] |
|---|---|
| 1:1 | 0 |
| 1:2 | 62.8 |
| 1:3 | 76.8 |
| 1:4 | 84.0 |
| 1:5 | 88.0 |

### Example 6

### Synthesis of Stearoyl Uridine with Water Removal by Azeotropic Distillation.

The synthesis of uridine esters was performed with azeotropic removal of the water produced. 25 g uridine (0.1 mol) was esterified with 58 g stearic acid (0.2 mol) in 150 ml of 2-methyl-2-butanol with 5 g Novozym 435 as biocatalyst. The reaction was performed at 60° C. with a vacuum of 110-120 mbar. The solvent/azeotrope was evaporated through a column. Azeotrope was collected on the top of the column. In the first 6 hrs of the reaction, azeotrope was stripped off from time to time. Afterwards, the reaction was carried out under total reflux conditions. Evaporated solvent/azeotrope was replaced by fresh solvent to keep a constant liquid level. Conversion was calculated based on the consumed amount of fatty acid.

| Reaction time [h] | Conversion [%] |
|---|---|
| 0 | 0.0 |
| 2.5 | 56.4 |
| 21.0 | 98.2 |

### Example 7

### Synthesis of Acyl Uridines by Transesterification from Oils/Triglycerides

The synthesis of acyl ribonucleosides was carried out with triglycerides. 2.5 g uridine (10 mmol) was transesterified with 5.1 g Myritol® 318 (mixture of C8/C10 triglycerides, 10 mmol) in 30 ml of 2-methyl-2-butanol with 0.5 g Novozym 435 as biocatalyst. The reaction was performed at 60° C. for 115 hrs. Conversion of uridine was calculated on GC analysis.

| Triglyceride | Conversion [%] |
|---|---|
| Myritol ® 318 | 30.0 |

### Example 8

### Synthesis of Uridine Stearate (Mono-O-Stearoyl Uridine)

The synthesis of uridine stearate was performed with 4.9 g uridine (20 mmol), 28.5 g stearic acid (100 mmol), 10 g Novozym 435, 30 g molecular sieves in 200 ml t-butanol. Uridine (No. 94320) was purchased from Fluka, Switzerland, stearic acid was from Cognis GmbH & Co. KG, Germany, molecular sieves (No. 1.057041.000) and t-butanol (No. 8.22264.1000) were from Merck KGaA, Germany. Novozym 435 was purchased from Novozymes A/S, Denmark. The reaction was carried out at 60° C. in a shaker. After 48 hrs reaction time, the solution was filtrated to remove the biocatalyst. The solution was evaporated and the product was subsequently extracted with hexane and water to the desired purity. After 2 extractions with hexane and 1 extraction with water, the purity of the uridine stearate (mono-O-stearoyl uridine) according to GC was higher than 70%.

### Example 9

### Synthesis of Uridine Palmitate (Mono-O-Palmitoyl Uridine)

The synthesis of uridine palmitate was performed with 4.9 g uridine (20 mmol), 14.9 g palmitic acid (60 mmol), 10 g Novozym 435, 30 g molecular sieves in 200 ml t-butanol. Uridine (No. 94320) was purchased from Fluka, Switzerland, palmitic acid was from Cognis GmbH & Co. KG, Germany, molecular sieves (No. 1.057041.000) and t-butanol (No. 8.22264.1000) were from Merck KGaA, Germany. Novozym 435 was purchased from Novozymes A/S, Denmark. The reaction was carried out at 60° C. in a shaker. After 48 hrs reaction time, the solution was filtrated to remove the biocatalyst. The solution was evaporated and the product was subsequently extracted with hexane and water to the desired purity. After 2 extractions with hexane and 1 extraction with water, the purity of the uridine palmitate (mono-O-palmitoyl uridine) according to GC was higher than 65.

### Example 9a

### Synthesis of Acetyl- an Butyryl-Uridine

O-acetyl-uridine and O-butyryl-uridine have been synthesized according to A Zinni et al., Biotechnoloy Letters 24, 2002, 979-983.

Analysis was performed by gas chromatography and thin layer chromatography.

| | Mono-ester | Di-ester | Tri-ester | Residue |
|---|---|---|---|---|
| | (%) | (%) | (%) | (%) |
| Mono/Di-O-Acetyl-Uridine | 60.2 | 33.7 | — | 6.1 |
| Tri-O-Butyryl-Uriidine | — | — | 84.3 | 15.7 |

In the following examples, the uridine used was purchased from Fluka, Switzerland (No. 94320), uridine stearate and uridine palmitate were synthesised and purified according to examples 8 and 9.

### Example 10

### Non-Toxicity of Acyl Ribonucleosides on Fibroblast Cultures In-Vitro.

In a first experiment, human fibroblasts were inoculated in a standard cell culture medium of foetal calf serum (or FCS). After an incubation of 1 day at 37° C. under an atmosphere of air, enriched to a carbon dioxide content of 5%, the growth medium was exchanged for a standard medium with a range of concentrations of uridine, uridine palmitate and uridine stearate. Uridine palmitate and uridine stearate were added to the culture medium starting from a stock solution at 1% (w/v) (1% w/v means 1 g of uridine etc in 100 ml solution) in DMSO. After an incubation of 3 days the number of viable cells was determined by evaluation of the levels of cellular proteins according to Bradford (Bradford M. M. A rapid and sensitive method for the quantification of microgram quantities ofprotein utilizing the principle of protein-dye binding, Anal. Biochem. (1977) vol 72, pp. 248 to 254) and the LD₅₀ was calculated. The results are presented in table 1.

In a second experiment, human fibroblasts were inoculated in a standard cell culture medium of foetal calf serum (or FCS). After an incubation of 3 days the cells became quiescent, then the growth medium was exchanged for a standard medium with a range of concentrations of uridine, uridine palmitate and uridine stearate. Uridine palmitate and uridine stearate were added in the culture medium starting from a stock solution at 1% (w/v) in DMSO. After an incubation of 3 days the number of viable cells was determined by evaluation of the levels of cellular DNA (fluorescent probe), ATP, proteins (Bradford's method) and the rate of cellular GSH (GSH is glutathione) normalised to the level of cellular proteins. The results are presented in table 2.

TABLE 1
LD₅₀ and level of cellular proteins in %/control (mean on 2 assays in
triplicate, i.e. 2 different assays were performed (two different fibroblast
cultures at 2 different times), each assay has been performed on three
different cell cultures in parallel (triplicate)).

| | | |
|---|---|---|
| LD₅₀ | Dose | |
| (% w/v) | (% w/v) | Proteins |

| | | | |
|---|---|---|---|
| Control | / | — | 100 |
| Uridine | >0.3% | 0.03 | 104 |
| | | 0.1 | 99 |
| Mono-O-palmitoyl uridine | >0.01% | 0.003 | 92 |
| | | 0.01 | 97 |
| Mono-O-stearyl uridine | >0.01% | 0.0005 | 90 |
| | | 0.0015 | 87 |

TABLE 2
Level of DNA, ATP, cellular proteins and GSH/protein in %/control
(mean on 2 assays in triplicate)

| | Dose | | | | GSH/ |
|---|---|---|---|---|---|
| | (% w/v) | DNA | ATP | Proteins | proteins |
| Control | — | 100 | 100 | 100 | 100 |
| Uridine | 0,01 | 103 | 100 | 100 | 107 |
| | 0,03 | 94 | 89 | 104 | 102 |
| Mono-O-palmitoyl uridine | 0,001 | 92 | 111 | 98 | 103 |
| | 0,003 | 83 | 86 | 91 | 108 |
| Mono-O-stearoyl uridine | 0,0015 | 94 | 91 | 96 | 98 |
| | 0,005 | 100 | 92 | 95 | 135 |

Up to the concentration of 0.01%, the acyl ribonucleosides uridine palmitate and uridine stearate have not shown any toxic effects on growing human fibroblasts cultured in vitro. They have not modified the growth of the fibroblasts, neither have they modified their energetic metabolism nor their protein metabolism.

### Example 12

### Inhibition of Melanin Synthesis.

Melanin is the pigment responsible for the colour of skin and hairs. Melanin synthesis takes place in specific organelles so called melanosomes in human melanocytes located in the basal layer of the human epidermis. This synthesis begins by oxidation of tyrosine to DOPA (dihydroxy-phenyl-alanine) by tyrosinase and then DOPA polymerises to melanin which is stored in melanosomes.

Melanocytes (B16 cell line: B16 is the name of the mouse melanoma cells used in this test.) were inoculated in standard cell culture medium of foetal calf serum (FCS). After an incubation of 3 days at 37° C. and CO2=5%, the growth medium was exchanged for a standard medium with a range of concentrations of uridine, uridine palmitate and uridine stearate. Uridine palmitate and ulidine stearate were added to the culture medium starting from a stock solution at 1% (w/v) in DMSO. After an incubation of 3 days, the number of viable cells was determined by the evaluation of the levels of cellularproteins (Bradford's method) and the level of synthesized melanin was measured by recording the optical density at 475 nm of cell's homogenate. The results are expressed in table 4.

TABLE 4
Results in % against control (mean on 2 assays in triplicate):

| | Dose | Rate of cellular | |
|---|---|---|---|
| | (% w/v) | proteins | Rate of Melanin |
| Control | | 100 | 100 |
| Uridine | 0.03 | 94 | 92 |
| | 0.1 | 97 | 92 |
| Mono/Di-O-Acetyl-Uridine | 0.001 | 106 | 108 |
| | 0.003 | 105 | 105 |
| | 0.01 | 102 | 86 |
| Tri-O-Butyryl-Uridine | 0.001 | 100 | 106 |
| | 0.003 | 106 | 106 |
| | 0.01 | 103 | 105 |
| | 0.001 | 100 | 98 |
| Mono-O-palmitoyl uridine | 0.003 | 95 | 69 |
| | 0.01 | 110 | 44 |
| | 0.001 | 98 | 84 |
| Mono-O-stearoyl uridine | 0.003 | 92 | 65 |
| | 0.01 | 99 | 19 |

The acyl ribonucleosides uridine palmitate and uridine stearate have strongly decreased the rate of released melanin at concentrations which have not shown any toxic effects on human fibroblasts cultured in vitro. On the opposite, uridine, uridine acetate and uridine butanoate have no or only a very poor effect on the inhibition of melanin synthesis.

1. A compound selected from the group consisting of an acyl ribonucleoside and an acyl deoxyribonucleoside wherein the acyl group or the acyl groups is/are derived from a fatty acid, (preferably an unsubstituted, linear, saturated or unsaturated carboxylic acid) with 10 to 20 (preferably 16 to 18) carbon atoms or from 3-phenyl-propionic acid or from 12-hydroxy-stearic acid or from octadecanoic diacid or from hexadecanoic diacid or from azelaic acid or from octadecenoic diacid, whereby in the case of octadecanoic diacid or hexadecanoic acid or azelaic acid or octadecenoic diacid one or both COOH groups of the acid can be esterified with a nucleoside.

2. A compound according to claim 1 selected from the group consisting of palmitoyl uridine, 5′-O-palmitoyl uridine, palmitoyl guanosine, palrnitoyl adenosine, palmitoyl cytidine, oleyl uridine, 5′-O-oleyl uridine, oleyl guanosine, oleyl adenosine, oleyl cytidine, stearoyl uridine, 5′-O-stearoyl uridine, 3-phenyl-propionyl uridine, the monoester of uridine with octadecanoic diacid, the diester of uridine with octadecanoic diacid, the monoester of uridine with hexadecanoic diacid, the diester of uridine with hexadecanoic diacid, the monoester of uridine with azelaic acid, the diester of uridine with azelaic acid, the monoester of uridine with octadecenoic diacid, the diester of uridine with octadecenoic diacid and 12-hydroxy-stearoyl uridine.

3. A composition comprising
3.1. an acyl ribonucleoside or an acyl deoxyribonucleoside having the following formulae I or II, 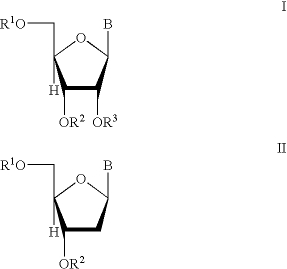
wherein
B is a nucleobase-moiety,
R¹, R² and R³ are independently selected from the group consisting of
a) hydrogen,
b) a saturated or unsaturated, linear or branched acyl radical with 3 to 22 carbon atoms, optionally substituted with one or more substituents selected from the group consisting of hydroxy, hydroxy-alkyl, amino, amino-alkyl, mercapto, mercapto-alkyl, halogen and thiolanyl,
c) a saturated or unsaturated, linear or branched dicarboxylic acid radical with 3 to 22 carbon atoms or its derivative in which the —-COOH-group that is not esterified with an OH-group of ribose or deoxyribose is replaced by —-CONR′₂ or by CONR′₃⁺S³¹ (wherein R′ is a hydrogen atom, a saturated or unsaturated, linear or branched alkyl radical with 1 to 6 carbon atoms, or an aryl radical, or an aralkyl radical or an aralkylene radical and wherein S⁻ a counter ion) or by COHal (wherein Hal is a halogen atom) or by COSH (wherein S is sulphur),
d) a saturated or unsaturated, linear or branched dicarboxylic acid diradical with 3 to 22 carbon atoms,
e) an arylaliphatic acid radical and derivatives thereof, optionally substituted with one or more substituents selected from the group consisting of hydroxy, nitro, alkyl, alkoxy and halogen and
f) a benzoic acid radical, optionally substituted with one or more substituents selected from the group consisting of hydroxy, nitro, alkyl, alkoxy and halogen
and wherein in the case of formula I at least one of the substituents R¹, R² and
R³ is not hydrogen and in the case of formula II at least one of the substituents R¹ and R² is not hydrogen and
3.2. auxiliaries and/or additives that are common for cosmetic purposes.

4. The use of the compound according to claim 1 or 2 or of the acyl ribonucleoside or the acyl deoxyribonucleoside as defmed in paragraph 3.1 of claim 3 or of the composition according to claim 3 for the manufacture of a cosmetic preparation.

5. The use of the compound according to claim 1 or 2 or of the acyl ribonucleoside or the acyl deoxyribonucleoside as defmed in paragraph 3.1 of claim 3 or of the composition according to claim 3 for the cosmetic treatment of the human body.

6. The use of the compound according to claim 1 or 2 or of the acyl ribonucleoside or the acyl deoxyribonucleoside as defined in paragraph 3.1 of claim 3 for the manufacture of a medicament for the treatment of human skin that has been damaged by UV-A radiation or by UV-B radiation or for the manufacture of a medicament for the treatment of inflammations of the human skin.

7. The use of the compound according to claim 1 or 2 or of the acyl ribonucleoside or the acyl deoxyribonucleoside as defmed in paragraph 3.1 of claim 3 as a food supplement.

8. A process for manufacturing the acyl ribonucleoside or the acyl deoxyribonucleoside as defined in paragraph 3.1 of claim 3 comprising reacting (optionally in a non-toxic solvent) the ribonucleoside or the deoxyribonucleoside with an acyl group donor in the presence of an enzymatic catalyst (optionally in soluble or in immobilised form).

9. The process of claim 8, wherein the acyl donor is the corresponding carboxylic acid.
